(19) United States
(12) Patent Application Publication
(10) Pub. No.: US 2002/0016603 A1
(43) Pub. Date: Feb. 7, 2002

(54) Surgical instrument with cushioned handle assembly

(76) Inventor: Timothy N. Wells, Ridgefield, CT (US)

Correspondence Address: Chief Patent Counsel, United States Surgical, Division of Tyco Healthcare Group LP, 150 Glover Avenue, Norwalk, CT, 06856, US

(21) Appl. No.: 09/865,776

(22) Filed: May 25, 2001

Related U.S. Application Data
(60) Provisional application No. 60/207,012, filed on May 25, 2000.

(51) Int. Cl. A61B 17/08

(52) U.S. Cl. 606/153

(57) ABSTRACT

A surgical instrument is provided which includes a cushioned portion attached to at least one of the pressure contact regions of the handle assembly. The cushioned portion is preferably formed from a material which is slip-resistant even in the presence of body fluids. The cushioned portion may be over-molded onto the pressure contact regions of the handle. Alternately, the cushioned portion can be fastened to the pressure contact regions of the handle assembly using other fastening techniques including adhesives, screws, welding, etc. In one preferred embodiment, the cushioned portion is formed from a thermoplastic elastomer. However, other slip-resistant pliant materials may also be used including elastomers, synthetics, etc.

This application claims priority from provisional application Ser. No. 60/207,012, filed May 25, 2000, which is incorporated herein by reference.

### BACKGROUND OF THE INVENTION

1. Technical Field

The present disclosure relates generally to surgical instrumentation having handle actuators. More particularly, the present disclosure relates to surgical instrumentation including a handle actuator for actuating an end effector supported on one end of a surgical instrument.

2. Background of Related Art

Surgical instrumentation for applying fasteners, e.g., staples and clips, to tissue and performing other tasks are well known in the art, and are commonly used to perform a variety of surgical procedures. Typically, these instruments include an end effector, e.g., an anvil, and a drive mechanism for actuating the end effector, e.g., driving a staple (or clip) or a plurality of staples through tissue and into contact with the anvil to deform the staples. The drive mechanism is actuated by a handle assembly which includes a movable actuator, e.g., a pivotable trigger, a slidable knob, a button, a rotatable knob, etc. The force required for a surgeon to grip the handle assembly and move the actuator to actuate the device varies greatly depending on the type of actuator and on the size and type of surgical instrument. Such force may be as high as about 50 lbs. or higher and is generally between about 20 lbs. and about 50 lbs.

Generally, the handle assemblies of surgical instruments are formed from hard plastic or surgical grade steel, e.g., stainless steel. During certain surgical procedures, a surgeon may be required to actuate the drive mechanism to actuate the end effector, e.g., fire staples or clips, a multiplicity of times causing the surgeon discomfort. Such repetitive use may eventually lead to bruising of the surgeon's hand and/or repetitive injury. It is common for blood and other body fluids to collect on the surgeon's gloves and on the instruments used by the surgeon during the surgical procedure. The presence of these fluids on the surgeon's gloves or hands and the surgical instruments make it difficult for the surgeon to grip and manipulate the surgical instruments.

Accordingly, a need exists for a surgical instrument which can be actuated by a surgeon multiple times without causing the surgeon discomfort. A need also exists for a surgical instrument which includes a handle assembly which can be easily gripped and manipulated by a surgeon during a surgical procedure even in the presence of blood and other bodily fluids.

### SUMMARY

In accordance with the present disclosure, a surgical instrument having an end effector and a handle assembly including an actuator for actuating the end effector is provided. A cushioned portion is attached to one or more pressure contact regions of the handle assembly. The cushioned portion is preferably formed from a material which is slip-resistant even in the presence of body fluids. In a preferred embodiment, the cushioned portion is over-molded onto the pressure contact regions of the handle assembly. Alternately, the cushioned portion can be fastened to the pressure contact regions of the handle assembly using other fastening techniques, e.g., physical, chemical or mechanical, including adhesives, screws, welding, etc. In one preferred embodiment, the cushioned grip portion is formed from a thermoplastic elastomer. However, other slip-resistant, pliant materials may also be used including thermoplastics, elastomers, synthetics, etc. Slip-resistance can be provided or enhanced by providing the cushioned portion with a textured or roughened surface finish. By providing a cushioned, slip-resistant portion on the operator pressure contact regions of the handle assembly, a surgeon can more easily grasp and manipulate the instrument with less or no discomfort.

### BRIEF DESCRIPTION OF THE DRAWINGS

Various preferred embodiments of the presently disclosed surgical instrument are described herein with reference to the drawings wherein:

FIG. 1 is a side view of one preferred embodiment of a stationary handle portion of the presently disclosed surgical instrument prior to attachment of the cushioned portion;

FIG. 2 is a side view of the stationary handle portion shown in FIG. 1 with the cushioned grip portion separated therefrom;

FIG. 3 is a side view of the stationary handle portion shown in FIG. 1 with the cushioned portion fastened thereto;

FIG. 4 is a perspective view of an end-to-end anastomosis instrument including cushioned portions positioned on the pressure contact regions of the handle assembly;

FIG. 5 is a perspective view of a gastrointestinal anastomosis instrument with cushioned portions positioned on the pressure contact regions of the handle assembly; and

FIG. 6 is a perspective view of a transverse anastomosis instrument with cushioned portions positioned on the pressure contact regions of the handle assembly.

### DETAILED DESCRIPTION OF PREFERRED EMBODIMENTS

Preferred embodiments of the presently disclosed surgical instrument will now be described in detail with reference to the drawings wherein like reference numerals designate identical or corresponding elements in each of the several views.

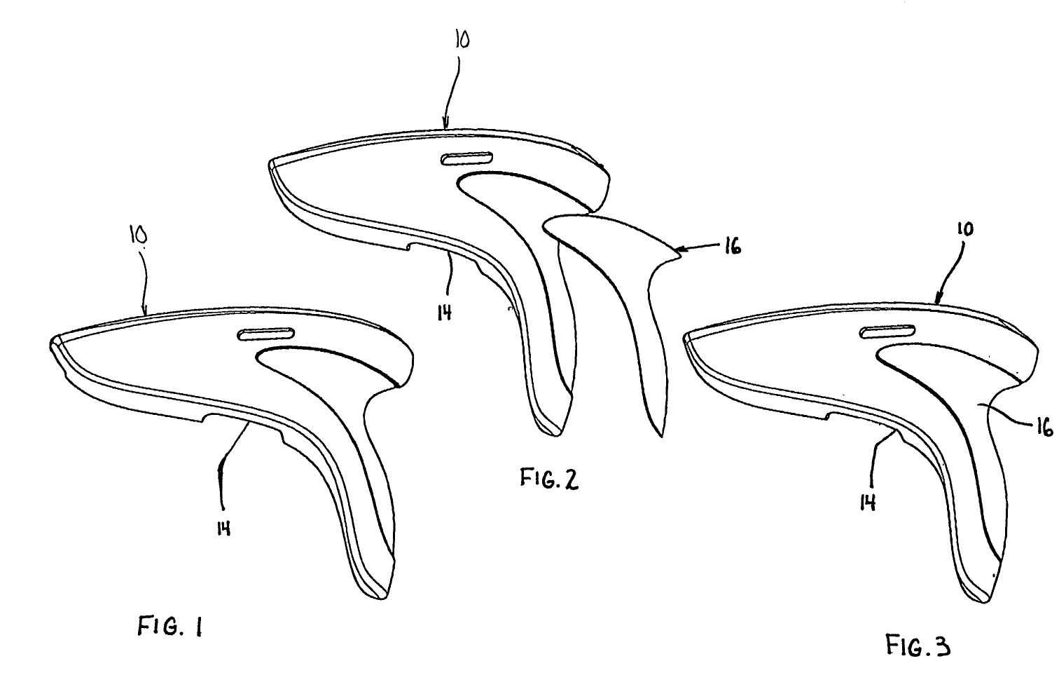

FIGS. 1-3 illustrate the stationary handle portion 10 of a surgical instrument. Stationary handle portion 10 includes a rigid or substantially rigid body portion 14 and a pliant or cushioned portion 16. In one preferred embodiment, stationary handle portion 10 is injection molded from a thermoplastic material and cushioned portion 16 is formed from a thermoplastic or thermoplastic elastomer blend, e.g., Versaflex™ or Santaprene™. A preferred thermoplastic elastomer is OM1040-X Versaflex™. A thermoplastic elastomer is a thermoplastic material that has rubber-like or flexible properties and can be injection molded. Alternately, body portion 14 can be formed from other materials including a variety of surgical grade metals and plastics and portion 14 can be formed from other pliant or elastomeric materials including isoprenes or nitrile or silicon containing materials, etc.

Cushioned portion 16 is fastened to the pressure contact regions of the handle assembly. The pressure contact regions include those areas of the handle assembly to which a surgeon must apply pressure, e.g., hand and/or digital, during manipulation and actuation of the surgical instrument. In a preferred embodiment, portion 16 is over-molded onto body portion 14. Alternately, portion 16 can be secured to body portion 14 using any known suitable physical, chemical or mechanical fastening techniques including adhesives, interengaging members, screws, welding, bonding, fusing, coating, dipping, spraying, etc.

The use of a cushioned portion formed from a thermoplastic elastomer or an elastomeric material on the pressure contact regions of the handle assembly cushions the impact on a surgeon's hand during operation of the surgical instrument. Preferably, the material is slip-resistant or is provided with a slip-resistant surface, e.g., roughened, textured, ridged, etc., such that the surface(s) adhere well to the gloves worn by a surgeon, even in the presence of bodily fluids, to improve a surgeon's grip on the surgical instrument.

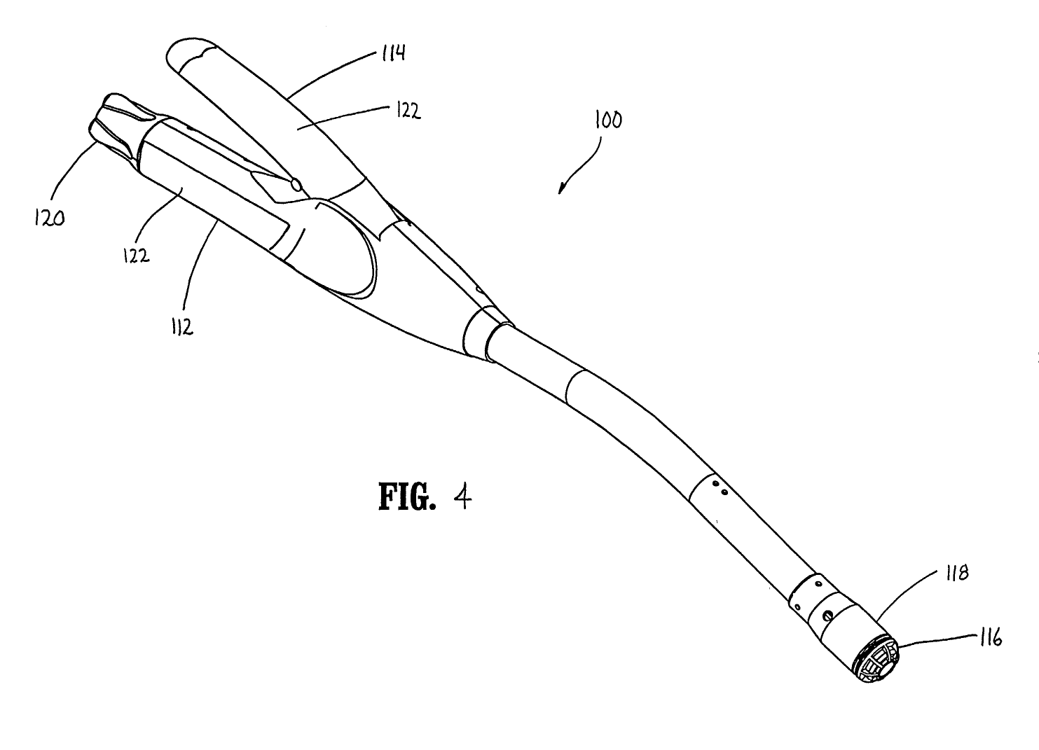

FIG. 4 illustrates a surgical stapler 100 including an end effector for performing end-to-end anastomosis. Stapler 100 includes a handle assembly including a stationary handle 112 and a pivotable handle 114. The end effector includes an anvil assembly 116 and a cartridge assembly 118 which houses a circular array of staples. A rotatable knob 120 is supported on the proximal end of stationary handle 112 and is rotatable to move cartridge assembly 118 in relation to anvil assembly 116 between spaced and approximated positions. Pivotable handle 118 and stationary handle 112 each include a cushioned member or layer 122 which is fastened to at least one of the pressure contact regions of the handle assembly. Actuator knob 120 may also be provided with a cushioned member in accordance with this disclosure. Preferably, cushioned layer 122 includes an over-molded thermoplastic elastomeric material. Alternately, the use of other pliant materials and fastening techniques, are envisioned.

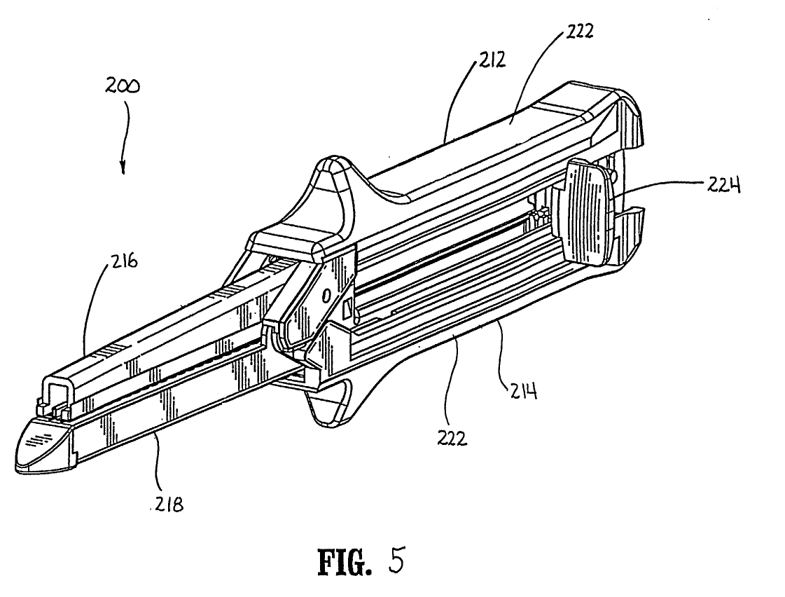

FIG. 5 illustrates a surgical stapler 200 for applying linear rows of staples to tissue. Stapler 200 includes a handle assembly including handles 212 and 214 and an end effector including an anvil assembly 216 and a cartridge assembly 218. A firing knob 224 is slidable from a proximal position to an advanced position to sequentially eject staples from the cartridge. Each handle includes a cushioned member or layer 222 formed on pressure contact regions of the handle. The firing knob 224 may also include a portion of cushioning material.

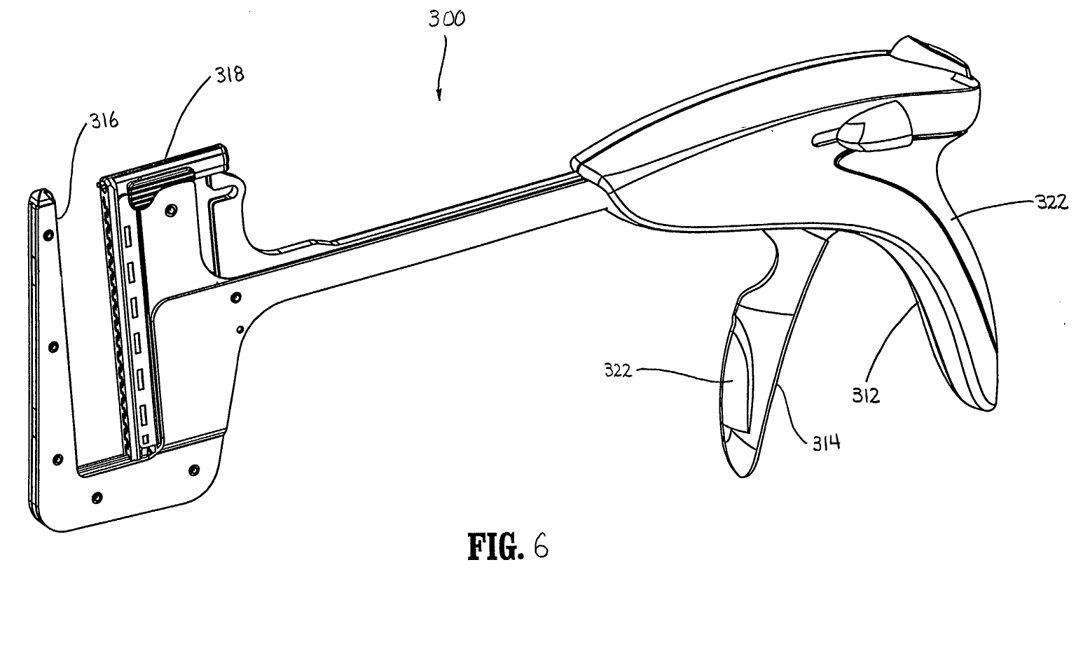

FIG. 6 illustrates a transverse anastomosis instrument 300 for applying linear rows of staples to tissue. Instrument 300 includes a handle assembly including a stationary handle 312 and a pivotable handle 314, and an end effector including an anvil assembly 316 and a cartridge assembly 318. Cartridge assembly 318 houses a plurality of rows of staples. Pivotable handle 314 is movable through an actuation stroke to move cartridge assembly 318 in relation to anvil assembly 316 between spaced and approximated positions. Handles 312 and 314 each include a slip-resistant, cushioned member or layer 322 formed thereon. Cushioned layers 322, as discussed above, can be over-molded about handles 312 and 314 or fastened thereto using any known fastening technique.

The hardness of the cushioning material employed will vary depending on the particular surgical instrument and its application. The pressure required to actuate a surgical instrument should be considered when choosing the material for forming the cushioned portion of the surgical instrument. For example, a softer material may be more suitable for use with instruments requiring higher actuation pressures. Conversely, a harder material may be suitable for use in surgical instruments requiring lower actuation pressures. The durometer of the cushioning material can be from about 10 to about 80, but is preferably from about 20 to about 50, and more preferably about 40.

Other factors should also be considered prior to selecting the cushioning material. These include whether the instrument is disposable or reusable and will be subjected to sterilization or other cleaning processes. If the instrument is reusable, a cushioning material having heat resistant properties should be used. In the alternative, it is contemplated that the cushioning member can be removable such that it could be removed from the surgical instrument prior to the sterilization and/or cleaning process. For example, the cushioning member could be provided as a removable member including a flexible sleeve.

The preferred method of providing surgical instruments with a cushioned portion to form the surgical instruments of the invention is to over-mold the cushioning material onto the desired portion(s) of the instrument. More particularly, the preferred method is a two-step injection molding process wherein, for example, by use of a rotary table, at a first station the plastic material for forming a rigid handle housing is injected into a mold in the form of the portion, e.g. the handle housing is injected into a mold in the form of the portion, e.g. the handle housing of the instrument to be over-molded. Once the plastic has cooled sufficiently, the mold is opened, and the table is rotated to move the formed handle housing to a second station. At the second station, the handle housing is placed in or is fully or selectively surrounded by a second mold. The cushioning material, preferably the themoplastic elastomer Versoplex™ OM 1040-X, is injected into the second mold such that the material is formed on the portion of the handle housing to be cushioned. After the cushioning material has sufficiently cooled and bonded or fused to the handle housing, the mold is opened and the finished cushioned handle is removed from the mold. Conventional over molding equipment, and conventional times temperatures and procedures can be employed in the two-step injection molding process. Though the above in-line process is preferred, other in-line or off-line injection over molding processes, or other forming processes can be employed.

It will be understood that various modifications may be made to the embodiments disclosed herein. For example, it is envisioned that other pliant or cushion materials may be used to achieve a cushioning effect similar to that disclosed above. Moreover, the above described pliant handle portion may be provided on other hand operated trigger devices such as graspers or cutting instruments. Therefore, the above description should not be construed as limiting, but merely as exemplifications of preferred embodiments. Those skilled in the art will envision other modifications within the scope and spirit of the claims appended hereto.

What is claimed is:

1. A surgical instrument comprising:
a handle assembly including a movable actuator;
a body portion extending distally from the handle; and
a head portion supported on one end of the body portion and including an end effector;
wherein the movable actuator includes a cushioned portion.

2. A surgical instrument according to claim 1, wherein the cushioned portion is formed from a thermoplastic elastomer.

3. A surgical instrument according to claim 1, wherein the cushioned portion is formed from an elastomeric material.

4. A surgical instrument according to claim 1, wherein the cushioned portion is over-molded onto the movable actuator.

5. A surgical instrument according to claim 1, wherein the movable actuator includes a pivotable trigger.

6. A surgical instrument according to claim 5, wherein the handle assembly further includes a stationary handle portion.

7. A surgical instrument according to claim 6, wherein the stationary handle portion includes a cushioned portion.

8. A surgical instrument according to claim 7, wherein the end effector includes a pair of jaws.

9. A surgical instrument according to claim 8, wherein one of the jaws includes a staple cartridge and the other of the jaws includes an anvil.

10. A surgical instrument according to claim 9, wherein the jaws are configured to perform end-to-end anastomoses.

11. A surgical instrument according to claim 8, wherein the jaws are configured to perform side-to-side anastomoses.

12. A surgical instrument according to claim 1, wherein the cushioned portion is formed from a slip-resistant material.

13. A surgical instrument according to claim 1, wherein the handle assembly includes a stationary handle portion, and the cushioned portion is positioned on the pressure contact regions of the movable actuator and the stationary handle portion.

14. A surgical instrument according to claim 1, wherein the cushioned portion is provided with a slip-resistant textured surface.

15. A surgical instrument according to claim 1, wherein the cushioned portion is a cushioned grip portion.

16. A process for forming a surgical instrument comprising the steps of:
injection molding a rigid handle housing;
placing the molded rigid handle housing adjacent an injection molding fixture configured to form a cushioning material at least one location on the molded rigid handle housing; and
injection molding a cushioning material on said at least one location on the molded rigid handle housing.

17. A process as in claim 16 wherein the step of injection molding a cushioning material includes injection molding a thermoplastic elastomer on said at least one location on the molded rigid handle housing.

18. A surgical instrument having a cushioning material formed thereon in accordance with the process of claim 16.

19. A process as in claim 16 wherein the steps of injection molding a rigid handle housing and injection molding a cushioning material on said handle housing are performed sequentially using a two shot injection process.
